(19) United States
(12) Patent Application Publication
(10) Pub. No.: US 2026/0177520 A1
(43) Pub. Date: Jun. 25, 2026

(54) ENZYME-BASED ELECTROCHEMICAL INLINE SENSOR WITH MEASURABLE SERVICE LIFE

(71) Applicant: Endress+Hauser Conducta GmbH+Co. KG, Gerlingen (DE)

(72) Inventors: Angela Eubisch, Chemnitz (DE); Michael Hanko, Dresden (DE); Martin Lohmann, Gerlingen (DE); Jörg Uhle, Limbach-Oberfrohna (DE); Jens Vettermann, Großweitzschen (DE)

(21) Appl. No.: 19/426,580

(22) Filed: Dec. 19, 2025

(30) Foreign Application Priority Data
Dec. 20, 2024 (DE) 10 2024 139 166.9
May 8, 2025 (DE) 10 2025 117 776.7
Sep. 29, 2025 (DE) 10 2025 139 397.4

(51) Int. Cl. G01N 27/327; C12Q 1/32; C12Q 1/34

(52) U.S. Cl. CPC G01N 27/3277; C12Q 1/32; C12Q 1/34; G01N 27/3273; G01N 2333/902; G01N 2333/90616; G01N 2333/98

(57) ABSTRACT

An electrochemical enzyme-based inline sensor for detecting a concentration of one or more analytes in a measuring medium, comprises at least one first electrode configured as a working electrode; at least one second electrode configured as a counter electrode, wherein the at least one first electrode, at which an enzyme-based redox reaction takes place, is configured to detect and transmit generated electrical charges from starting operation of the inline sensor and to generate a charge analyte-dependent current flow resulting from the redox reaction; and an evaluation device. The evaluation device is embodied to determine the concentration of the one or more analytes from the generated charges; and determine a measurand from the generated charges, wherein the measurand is used to determine an enzyme activity at the at least one first electrode, wherein the inline sensor is embodied to be installed in a process vessel and to be subsequently heat-sterilized.

### CROSS-REFERENCE TO RELATED APPLICATION

The present application is related to and claims the priority benefit of German Patent Application No. 10 2024 139 166.9, filed on Dec. 20, 2024, German Patent Application No 10 2025 117 776.7, filed on May 8, 2025, German Patent Application No. 10 2025 139 397.4, filed Sep. 29, 2025, the entire contents of which are incorporated herein by reference.

### TECHNICAL FIELD

The present disclosure relates to an inline sensor for the detection of measured values of an analyte content or a measurand representing an analyte content of a measuring medium. The present disclosure further relates to a method for the detection of measured values.

### BACKGROUND

In order to determine the composition of measuring media—for example, liquids, e.g., pure liquids, liquid mixtures, emulsions, or suspensions—various analytical measuring devices are used in process metrology and analysis metrology. An analytical measuring device generally comprises a sensor which is designed to generate an electrical measurement signal dependent upon at least one analytical measurand, as well as an electronic evaluation device which, from the measurement signal, determines a measured value representing the current value of the at least one analytical measurand in the measuring medium. The analytical measurand can, for example, be a concentration or activity of an analyte or a parameter dependent upon a concentration or activity of at least one analyte in the measuring medium.

An analyte here means one or more substances contained in and, for example, dissolved in the measuring medium, whose concentration in the measuring medium is to be determined or monitored by the sensor. The electronic evaluation device can be at least partially integrated into a measuring transducer arranged directly at the measurement point, which transducer has an electronics housing with display and input elements. At least one part of the electronic evaluation device may also be arranged together with the sensor in a common sensor housing.

Such analytical measuring devices are used in a variety of fields, e.g., for monitoring and controlling processes in pharmaceutical, chemical, biotechnical, or biochemical production, but also in processes for water conditioning or sewage purification, and in environmental analysis. Insofar as an analytical measuring device is used in a process, the measuring medium will typically be contained in a process vessel. Such a process vessel may, for example, be a pipeline of a process plant or a reaction vessel, for example a bioreactor or a fermenter.

A bioreactor, often also frequently called a fermenter, is a container in which certain microorganisms, cells or small plants are cultivated under highly optimal conditions.

Sensors that are integrated into the wall of a process vessel for monitoring a measurand of a measuring medium contained in the process vessel are referred to as inline sensors. An inline sensor detects the measurand directly in the measuring medium to be monitored. With inline sensors, an extraction and pre-treatment of a sample from the process is therefore not necessary for determining an analytical measurand. For the integration of a sensor into the process wall, diverse adapters and fittings—for example, immersion or retractable fittings—are known. An arrangement that comprises an inline sensor integrated into the wall of a process vessel and, if applicable, a measuring circuit and electronic evaluation device connected to the inline sensor, but offset from this, is referred to as an inline sensor arrangement. The inline sensor may be fixed in the wall by means of a suitable adapter.

With processes that must be implemented under sterile or aseptic conditions—for example, as they occur in biotechnology, pharmaceutics, or food technology—all parts of the process plant that come into contact with the process media—for example, all process vessels and also the sensors integrated therein—are typically sterilized (for example, thermally sterilized by heat) before the beginning of the process or between individual process steps. The heat sterilization may take place via dry heat or via superheated steam as a sterilization medium under increased pressure, e.g., via autoclaving in a pressure vessel, what is known as an autoclave. For example, superheated steam sterilization methods are common in which temperatures of at least 120° C. or more may occur. If the heat sterilization is performed in an autoclave, the process-contacting parts of the process plant—possibly already connected to one another—are introduced into the autoclave and are sterilized there. The sterilized parts are subsequently removed from the autoclave again and placed in operation.

Alternatively, a process plant may be sterilized by means of what is known as an SIP method (SIP is the acronym for the English technical term, “sterilization in place”), in which the process vessel to be sterilized, including the inline sensor integrated therein, is sterilized with superheated steam that is introduced into the process vessel over a predetermined time period. Therefore, inline sensors must be able to withstand the conditions that thereby occur, such as high temperatures of at least 120° C. and increased pressures, without loss of functionality.

In bioprocess metrology, e.g., for monitoring, controlling, and/or regulating biotechnology processes, sensors are also used that have immobilized biological detection elements on the surface of a sensor element, for example those that—possibly as receptors—selectively and specifically bind to and/or convert the analyte. Biological detection elements may be proteins such as enzymes or antibodies, DNA/RNA fragments, cell organelles, or whole cells and microorganisms. Such sensors are designated as biosensors.

After a typical, superheated, steam sterilization process, the receptors or biological detection elements of such biosensors, for example, proteins, cell organells or microorganisms are normally present with starkly reduced activity-most often, irreversibly denatured, i.e., their native three-dimensional structure is destroyed. Such biosensors therefore, in principle, may not be used as inline sensors in the wall of a process vessel and be sterilized with this by means of a typical SIP method, without taking further measures.

Sterilizable biosensors based upon amperometric enzyme sensors are described in the literature. In M. Phelps, Development of a regenerable glucose biosensor probe for bioprocess monitoring, master's thesis, University of British Columbia, 1993, an overview of the literature regarding such sensors is given. Strategies described therein for ensuring sterilization capability of such biosensors while maintaining their functionality include the introduction of the temperature-sensitive receptors arranged on a carrier—said carrier comprising, for example, a working electrode—only after the sterilization process in a reaction chamber within a sensor housing which is sealed in relation to the process vessel by a membrane permeable to the respective analyte. The membrane in this instance represents the sterile barrier. The receptors may thereby be present immobilized on the subsequently introduced working electrode, or in a solution received into the reaction chamber. The sterile barrier may not be destroyed upon introduction of the receptors, which makes the handling of such inline sensors difficult.

In addition to the thermal sensitivity of the sensor elements due to the risk of enzyme denaturation, the stability of the measurement signal when used in a bioprocess is a crucial parameter that is required for successful and reliable process monitoring.

In bioprocess engineering, the available amount of analytes such as glutamate or analytes that are formed as nutrients, such as glucose, is determined, among other things.

For example, to measure glucose, a sensor element is used in which the enzyme glucose oxidase (GOD) is immobilized on the working electrode. The glucose is converted by the enzyme as follows:

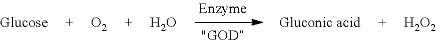

The resulting product, hydrogen peroxide, is oxidized amperometrically at a fixed potential on a platinum anode. In addition to molecular oxygen, two protons and two electrons are also produced. The resulting electrons (also charges in general) form the measurement signal and are directly proportional to the concentration of glucose. The increase in the hydrogen peroxide concentration or the decrease in oxygen concentration is recorded amperometrically, depending on the choice of polarization potential.

Catalytic effectiveness, and therefore enzymatic activity, are among the most important prerequisites for the functionality of an enzyme-based electrochemical sensor.

As a protein and biomolecule, each enzyme has a limited service life due to various deactivation mechanisms; that is, the number of catalyzed reactions per molecule is limited. The dimensionless “total turnover number” serves as a parameter. It is defined as the “number of catalytic events carried out by an active site of a molecule of the enzyme during its service life.”

A defined amount of enzyme is immobilized per sensor. Accordingly, the total number of generated electrons by one and the same sensor is a measure of the enzyme state or the remaining service life of the sensor. The total number or total quantity of generated electrons can be easily determined by means of a charge counter.

The total number or total quantity of generated electrons is therefore:
directly proportional to the working temperature of the enzyme=temperature of the customer process/measurement solution
directly proportional to the concentration of the measurement solution as well as the amount of enzyme on the sensor.

The total number or total quantity of generated electrons is a measure of the amount of converted analyte molecules.

The use of enzyme-based electrochemical sensors is currently limited just to analyses outside the process vessel—however, removal from the process vessel poses a risk of contamination. Furthermore, the measured values are determined in real time.

It is therefore desirable to integrate inline bioprocess sensors into a bioprocess that allow the measurement of analytes such as glucose without having to remove a sample from the process vessel.

### SUMMARY

The object of the present disclosure is therefore to provide an inline enzyme-based sensor with the possibility of monitoring the enzyme state in order to be able to assess the quality of the measurement.

The object is achieved by
an electrochemical enzyme-based inline sensor (1) for detecting the concentration of one or more analytes in a measuring medium (2), comprising:
   at least one first electrode which is designed as a working electrode, at least one second electrode which is designed as a counter electrode,
   in certain embodiments, a third electrode which is designed as a reference electrode, wherein
the at least first electrode, at which the enzyme-based redox reaction takes place, is formed,
to detect and transmit the generated electrical charges, for example, electrons, from the start of operation of the inline sensor, and
to generate an electron analyte-dependent current flow resulting from the redox reaction, as well as
   an evaluation device that is designed in such a way as
to determine the concentration of the analyte or analytes from the generated charges, for example, electrons, and
to determine a measurand derived from the generated charges, in order to determine the enzyme activity on the at least first electrode, wherein the inline sensor is designed to be installed in a process vessel, for example, process vessels for the production of biotechnological or biopharmaceutical products, and to be subsequently heat-sterilized.

For example, the inventive electrochemical enzyme-based inline sensor is used to determine:
(i) whether an enzyme-based inline sensor can be reused for a batch,
(ii) when a new sensor element or measuring electrode needs to be inserted and/or
(iii) the sensor quality at the end of the batch.

The enzyme activity of the enzymes applied to the inline sensor is monitored. Therefore, statements can be derived which have the following advantages:

Controlled insertion/or non-insertion prevents an inline sensor that is still capable of measurement from being deemed no longer valid and replaced too early. This allows the used inline sensor to be used optimally in the process. The present disclosure can reduce effort and costs. For example, the insertion is achieved by axial displacement relative to the outer tube of the inline sensor.

The statement regarding sensor quality at the end of a batch can be part of the release process during manufacturing; if it is known early on that the inline sensor can no longer measure validly, the customer can identify timely countermeasures.

In at least one embodiment, the measurand derived from the generated charges is formed from the comparison of the amount of charge integrated over time and a reference signal that represents the maximum amount of charge of the inline sensor and is a value stored in the inline sensor (1), or
from the comparison of the current flow resulting from the generated charges and a reference current flow.

In at least one embodiment of the electrochemical enzyme-based inline sensor,
the first electrode comprises a sensor element, wherein the first electrode comprises a sensor element and is surrounded by at least one housing, wherein the at least one housing is designed to enclose the sensor element during heat sterilization in a chamber sealed gas-tight from an environment or medium by means of at least one first sealing element and, after heat sterilization, to bring it into sterile contact with the sterilized environment and/or medium to be measured in a measuring position.

In at least one embodiment of the aforementioned electrochemical enzyme-based inline sensor, the at least one housing has:
an outer tube (7) and a cavity (8) enclosed by the outer tube (7),
an inner tube (9), and
-a carrier (10) which is permanently connected to the inner tube (9), for example, arranged on the medium side during operation, wherein the first electrode (3) comprising a sensor element (4) is permanently connected to the carrier, wherein
the carrier (10) is positioned in an irradiation position with sterilizing radiation, for example, gamma radiation, in the cavity of the outer tube (8) such that at least the chamber (6) is arranged between the carrier (10) and the cavity of the outer tube (8), wherein the chamber (6) is designed as a gap (6) which is designed such that, in the irradiation position, the sensor element (4) arranged in the carrier (9) is in contact with the environment (2), for example, in gas contact, and the carrier (10) is positioned in the hot steam sterilization position such that the medium-side end section of the carrier (10) seals the sensor interior and the sensor element (4) arranged in the carrier (10) gas-tight against the environment (2) of the inline sensor (1) by a first sealing element (13).

In at least one embodiment of the aforementioned electrochemical enzyme-based inline sensor, the carrier and the sensor element are positioned in the measuring position such that the sensor element is designed to be in contact with the environment, for example, with the measuring medium, after hot steam sterilization.

In at least one embodiment of the two aforementioned electrochemical enzyme-based inline sensors, the carrier (8) permanently connected to the inner tube and containing the at least one sensor element is movable unidirectionally in the direction of the measuring medium in operation relative to the outer tube along the longitudinal axis (L) of the sensor from an irradiation position to a hot steam sterilization position and from the hot steam sterilization position to a measuring position, and can be locked in the hot steam sterilization position and the measuring position.

In at least one embodiment of the three aforementioned electrochemical enzyme-based inline sensors,
(i) the outer tube
   has a substantially cylindrical cavity,
   at least one first connecting element on an outer wall at the end facing away from the medium
   a third connecting element on an inner wall,
and
   at least one, for example, a first and at least one second, sealing element on the inner wall at the end facing the medium;
(ii) the inner tube
   has a fourth connecting element on an outer wall, which is designed to engage in a third connecting element on the inner wall of the outer tube in the measuring position, and
   is connected at its end facing away from the medium to a sheath, wherein the inside of the sheath has at least one second connecting element which is designed to allow one or more first connecting elements to snap into the outer wall of the outer tube in the hot steam sterilization position,
   an electronics housing connected to the inner tube in a gas- and liquid-tight manner, which is connected to a locking element in a gas- and liquid-tight and detachable manner, wherein the locking element consists of a terminal removable, for example, unscrewable, transport lock or comprises same, wherein the transport lock is designed in the installed state to prevent accidental and unintentional transfer of the inner tube from the hot steam sterilization position to the measuring position and to release the movement after removal;
wherein the end of the inner tube released by the transport lock has at least one overhang which is designed to prevent movement of the inner tube and the associated carrier containing the sensor element beyond the measuring position; and
(iii) the carrier is permanently connected, with a section of its outer shell facing away from the medium, to a medium-side section, for example, to a medium-side end section, of the inner surface of the inner tube.

The maximum amount of charge depends, among other things, on how long the inline sensor was heat sterilized. The maximum amount of charge is therefore defined as the amount of charge that the electrochemical inline sensor has in the measuring medium before starting operation. This amount of charge represents the maximum amount of charge of the sensor.

In at least one embodiment, the enzyme is selected from
an enzyme that catalyzes the oxidation of lactate, for example, a lactate oxidase,
an enzyme that catalyzes the oxidation of glutamate, for example, a glutamate oxidase,
an enzyme that catalyzes the oxidation of glutamine, for example, a glutaminase or
an enzyme that oxidizes glucose, for example, a glucose oxidase.

In at least one embodiment, the inline sensor is designed as an amperometric inline sensor.

In at least one embodiment, the electrochemical inline sensor contains at least two measuring electrodes comprising a sensor element, wherein the inline sensor is designed such that the electrodes, each comprising a sensor element, can be introduced into the measuring medium simultaneously or successively.

In at least one embodiment, the inline sensor further has a temperature sensor, wherein the temperature sensor is arranged within the sensor housing of the inline sensor or is a separate temperature sensor electrically connected to the inline sensor.

The sensor housing includes the outer tube, the inner tube, the connection, the sheath and the electronics housing of the inline sensor.

In at least one embodiment, the inline sensor at the end region, facing away from the medium of the medium-side part, of the outer tube 5 comprises a thread 24, for example a PG 13.5 thread 24, for installing the inline sensor 1 in a fitting, a process vessel, pipeline or a fermenter.

The present disclosure also relates to an analysis system for process monitoring of a process or a bioprocess, comprising:
a reaction vessel comprising a cell culture contained in a medium, for example, a suspension culture,
at least one electrochemical inline sensor, for example, two or more than two electrochemical inline sensors according to the present disclosure or an embodiment thereof.

The present disclosure also relates to a method for continuously determining the total enzyme activity of an electrochemical enzyme-based, inline sensor, comprising the steps of:
(i) providing an inline redox enzyme-based electrochemical inline sensor for process measurement technology or for bioprocess measurement technology to continuously monitor the state of the enzyme, comprising
   at least one sensor element comprising an amount of an immobilized redox enzyme, wherein the sensor element is electrically coupled to a first electrode and/or is connected thereto, which is designed as a working electrode,
   a counter electrode
   in certain embodiments, a reference electrode,
(ii) time-dependent determining of the charges received at the measuring station in the form of a current flow,
(iii) evaluating the current flow with an evaluation device to determine the concentration of the analyte in the liquid measuring medium, and
to determine the total enzyme activity of the inline sensor via a measurement quantity derived from the current flow, wherein the inline sensor (1) is designed to be installed in a process vessel, for example, process vessels for the production of biotechnological or biopharmaceutical products, and to be subsequently heat-sterilized.

In certain embodiments, the inline sensor (1) is the inline sensor (1) according to the present disclosure or an embodiment thereof.

In at least one embodiment of the method according to the present disclosure, the inline sensor further comprises a temperature sensor, wherein the temperature sensor is arranged within the sensor housing of the inline sensor or is a separate temperature sensor electrically connected to the inline sensor.

In at least one embodiment of the above-mentioned method, the measurand derived from the current flow is determined, wherein:
(i) the current flow at the working electrode is converted into a sensor signal correlated with the amount of charge and compared with a reference signal that represents the maximum amount of charge of the inline sensor, or
(ii) the measured current flow is compared with a measured reference current.

In at least one embodiment, the redox enzyme-based inline sensor is selected from an electrochemical inline sensor according to the present disclosure or an embodiment thereof.

In at least one embodiment of the method,
a drop in current flow relative to the reference current or
a saturation of the signal correlated with the amount of charge relative to a reference signal represents a drop in the enzyme state of the inline sensor.

In at least one embodiment of the method, when the current flow drops relative to the reference current or when the signal correlated with the amount of charge becomes saturated relative to a reference signal, another working electrode is introduced into the medium to be measured.

In at least one embodiment according to any of the preceding embodiments of the method,
a pre-alarm and/or an alarm is triggered at 90% or less than 90%, for example, 80% of the reference signal, or
at 90% or less than 90%, for example, 80% of the maximum current flow, which indicates a replacement of the working electrode or the sensor element.

The pre-alarm in this case is a warning to the user about the occurrence of a drop in current flow or saturation of the signal correlated with the amount of charge relative to a reference signal.

### BRIEF DESCRIPTION OF THE DRAWINGS

In the figures:

FIGS. 1a, 1b, and 1c show an embodiment of a sterilizable inline sensor;

FIG. 2 shows the maximum number of charge carriers at different glucose concentrations;

FIG. 3 shows a graphical representation of the drop in the sensor current.

### DETAILED DESCRIPTION

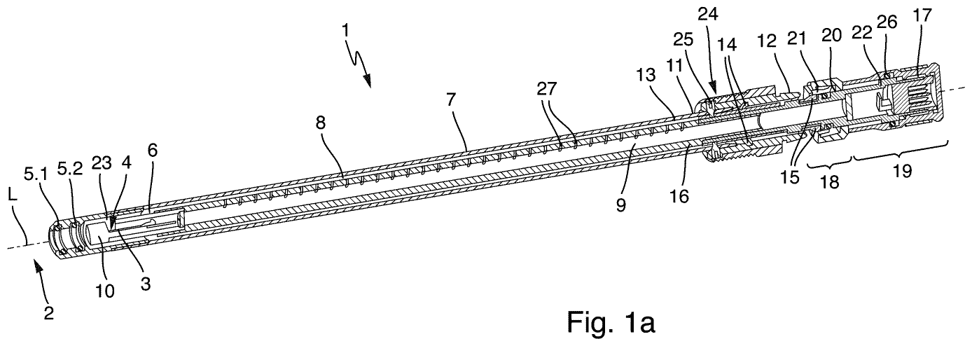

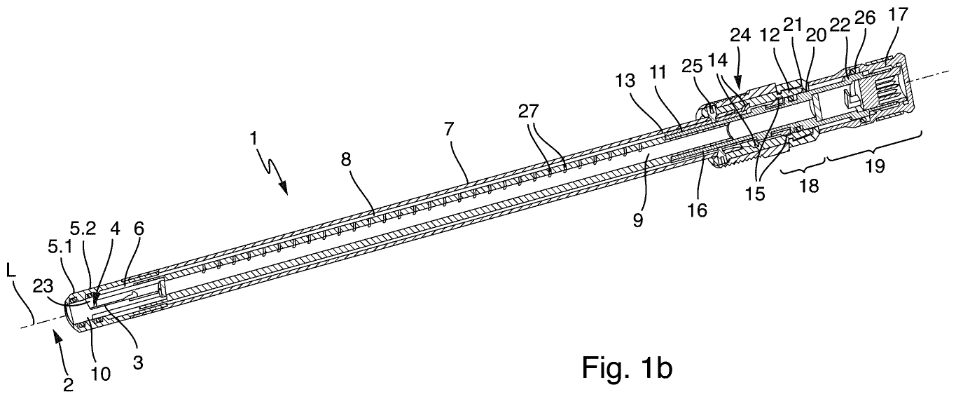

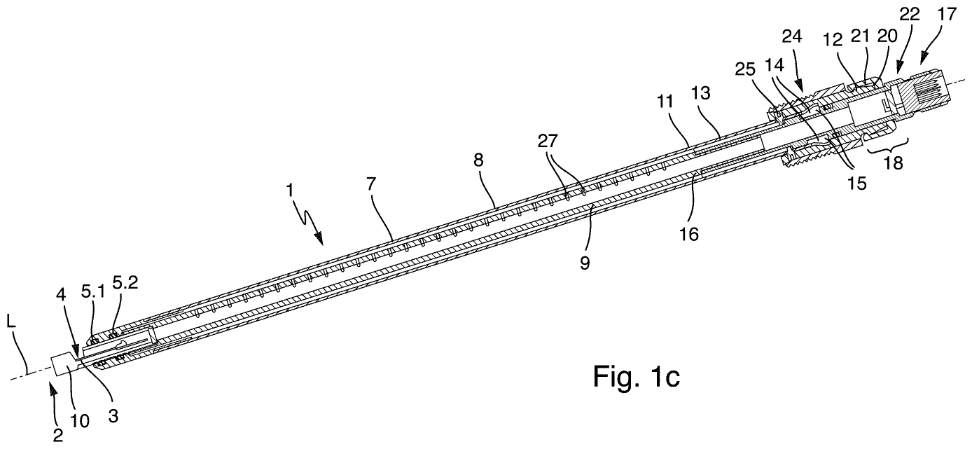

FIGS. 1a, 1b, and 1c show a sterilizable embodiment of the inline sensor according to the present disclosure. The inline sensor is designed to be gamma-sterilizable and heat-sterilizable. The inline sensor can assume three positions: a gamma sterilization position, a heat sterilization position, and a measuring position.

FIG. 1a shows an inline sensor 1 which substantially comprises an inner tube 9 and an outer tube 7. The inner tube 9 is mounted to be axially movable within the outer tube 7 along the longitudinal axis of the inline sensor. The anti-rotation devices 25 prevent a rotational movement of the inner tube 9 relative to the outer tube 7 around the longitudinal axis of the inline sensor. The inner tube 9 is permanently, for example, positively, connected with its medium-side or process-side end to an end of the carrier 10 facing away from the medium or facing away from the process, wherein the carrier 10 comprises the sensor element 4. The sensor element 4 is arranged in a recess 23 of the carrier 10. In the irradiation position, the inner tube 9, the carrier 10 associated therewith and the sensor element 4 comprised by the carrier 10 are located facing away from the medium or process from the first and second sealing elements 5.1 and 5.2. This creates a gap 5 between the carrier 10 and the outer tube 9 as well as between the inner tube 9 and outer tube 7, which allows the interior of the inline sensor to be filled with a protective gas, for example argon gas, and sterilized by radiation in the irradiation position. The inner tube is also gas-tight, for example, permanently and/or positively connected at the end facing away from the medium to an electronics housing 17 and includes an electrical connection to the electronics housing 17. The outer side of the inner tube is connected at the end facing away from the medium to a sheath 18 and a detachably connected locking element 19. The locking element 19 is embodied as a transport lock 19, wherein the locking element 19 comprises an anti-rotation device 19, for example a thread 19. A third sealing element 26 is arranged between the transport lock 19 and the electronics housing 17, wherein this sealing element 26 is, for example, designed as an O-ring. The inline sensor 1 has a medium-side or process-side part and a part facing away from the medium or facing away from the process. At the end region facing away from the medium of the medium-side part, the outer tube 5 comprises a thread 24, for example a PG 13.5 thread 24, for installing the inline sensor 1 in a fitting, a process vessel, pipeline or a fermenter. If the thread 24 is designed as an external thread, the fitting, the process vessel, a pipeline or fermenter has a corresponding internal thread. The medium-side or medium-contacting part has a length similar to that of common inline sensors for process automation, i.e., approximately 120 mm, 225 mm or 360 mm. The inline sensor is packaged in at least one, for example, two, flexible and radiation-sterilizable packages and sterilized by means of radiation that is suitable for disinfection or sterilization, wherein the radiation is UV, X-ray, gamma radiation or electron radiation. In at least one embodiment, a temperature sensor (not shown) is installed in the sensor housing of the inline sensor (not shown). The sensor housing includes the outer tube 5, the connection 24, the sheath 19 and the electronics housing 17.

Alternatively, the inline sensor is electrically connected to a separate temperature sensor that is designed to detect the temperature of the process medium.

FIG. 1b shows an inline sensor 1 in the hot steam sterilization position. The carrier 8 is enclosed in this case on the medium side by the first sealing element 13, for example a sealing ring, whereby the interior of the inline sensor, which is located facing away from the medium from the sealing ring, is sealed gas-tight from the environment. The transition to the heat sterilization position, for example, the hot steam sterilization position, is effected on the side facing away from the medium via the first connecting elements 10, which are positively attached to the outer wall 9 of the outer tube 5 and the second connecting elements 20, which are positively attached to the inside of the sheath 20. The inline sensor 1 is removed from the at least one radiation-sterilized package after being transferred to the heat sterilization position, for example, hot steam sterilization position, and the connection 24, for example, a thread 24, is installed in the container to be sterilized. The connection 24 is, for example, installed in a force-fit manner on the outer wall 9 of the outer tube 5. In the hot steam sterilization position, the sensor element is insulated against the hot steam sterilized vessel in a gas- and liquid-tight manner. The openings 27 are designed to establish gas contact between the sensor element and a desiccant.

FIG. 1c shows an inline sensor 1 in the measuring position. The sensor is transferred into the measuring position after heat sterilization, for example, hot steam sterilization. In order to allow the inner tube to be moved further axially towards the measuring medium 2 relative to the outer tube 7, the locking element 19, for example, the transport lock 19, is removed. Locking along the longitudinal axis of the inline sensor 1 is achieved through the interaction of the third connecting elements 14 and the fourth connecting elements 15.

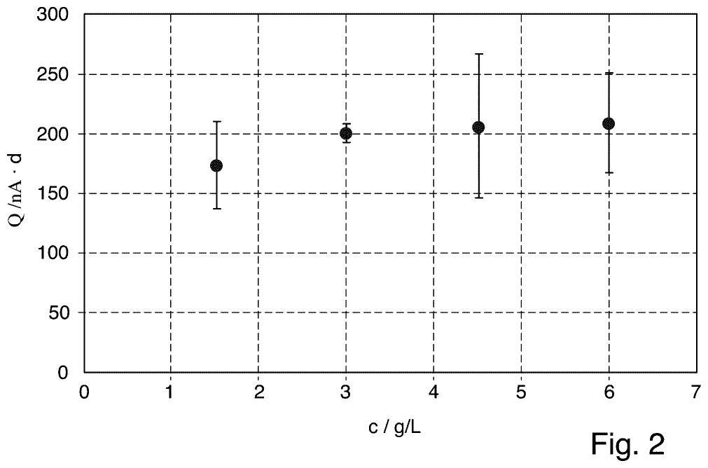

FIG. 2 shows that the maximum achievable amount of charge is constant in the range from 1 g/L to 6 g/L so that in this concentration range, there is no dependence on the glucose concentration.

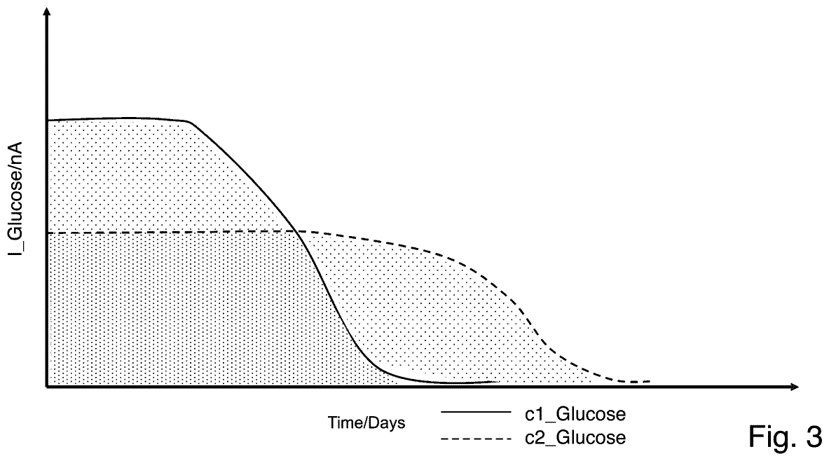

FIG. 3 shows a plot of the measured current over time for two different glucose concentrations, c1 and c2. The areas under the shown curves correspond to the amount of charge. FIG. 3 shows that the amount of charge that results from the integral of the measured current over time at concentrations c1 (=6 g/L) and c2 (=3 g/L) is independent of the concentration.

All above-described embodiments of the electrochemical enzyme-based inline sensor 1 and the method for continuously determining the total enzyme activity of an electrochemical enzyme-based inline sensor 1 can each be combined with each other, provided that this is technically possible.

Reference signs are not to be understood as a limitation of the scope of the subject matter protected by the claims. They serve only the purpose of making the claims easier to understand.

1. An electrochemical enzyme-based inline sensor for detecting a concentration of one or more analytes in a measuring medium, comprising:
at least one first electrode configured as a working electrode;
at least one second electrode configured as a counter electrode, wherein
the at least one first electrode, at which an enzyme-based redox reaction takes place, is configured to detect and transmit generated electrical charges from starting operation of the inline sensor and to generate a charge analyte-dependent current flow resulting from the redox reaction; and
an evaluation device embodied to:
determine the concentration of the one or more analytes from the generated charges; and
determine a measurand from the generated charges, wherein the measurand is used to determine an enzyme activity at the at least one first electrode, wherein the inline sensor is embodied to be installed in a process vessel and to be subsequently heat-sterilized.

2. The electrochemical enzyme-based inline sensor according to claim 1, wherein the measurand determined from the generated charges is determined by:
a comparison of an amount of charge integrated over time and a reference signal that represents a maximum amount of charge of the inline sensor, and a value stored in the inline sensor, or
a comparison of the current flow resulting from the generated charges and a reference current flow.

3. The electrochemical enzyme-based inline sensor according to claim 1, wherein the at least one first electrode comprises a sensor element and is surrounded by at least one housing, wherein the at least one housing is embodied to enclose the sensor element during heat sterilization in a chamber sealed gas-tight from an environment or a medium using at least one first sealing element and, after heat sterilization, to bring the sensor element into sterile contact with the sterilized environment or medium to be measured in a measuring position.

4. The electrochemical enzyme-based inline sensor according to claim 3, wherein the at least one housing comprises:
an outer tube;
a cavity enclosed by the outer tube;
an inner tube; and
a carrier permanently connected to the inner tube, wherein the at least one first electrode comprising a sensor element is permanently connected to the carrier, wherein
the carrier is positioned in an irradiation position with sterilizing radiation in the cavity of the outer tube such that at least the chamber is arranged between the carrier and the cavity of the outer tube, wherein the chamber is embodied as a gap such that, in the irradiation position, the sensor element arranged in the carrier is in contact with the environment, and the carrier is positioned in a hot steam sterilization position such that a medium-side end section of the carrier seals a sensor interior and the sensor element arranged in the carrier gas-tight against the environment of the inline sensor by the at least one first sealing element.

5. The electrochemical enzyme-based inline sensor according to claim 4, wherein the carrier and the sensor element are positioned in the measuring position such that the sensor element is in contact with the environment after hot steam sterilization.

6. The electrochemical enzyme-based inline sensor according to claim 4, wherein
the outer tube comprises:
a substantially cylindrical cavity;
at least one first connecting element on an outer wall at an end facing away from the medium;
a third connecting element on an inner wall; and
at least one sealing element on the inner wall at an end facing the medium, and the inner tube comprises:
a fourth connecting element on an outer wall, wherein the fourth connecting element is embodied to engage a third connecting element on the inner wall of the outer tube in the measuring position, and is connected at an end facing away from the medium to a sheath, wherein an interior of the sheath includes at least one second connecting element embodied to allow at least one first connecting element to snap into the outer wall of the outer tube in the hot steam sterilization position;
an electronics housing connected to the inner tube to be gas- and liquid-tight, and is connected to a locking element to be gas- and liquid-tight and detachable, wherein the locking element comprises a terminal removable unscrewable transport lock, wherein the transport lock in an installed state is embodied to prevent accidental and unintentional movement of the inner tube from the hot steam sterilization position to the measuring position and to release movement after removal, wherein an end of the inner tube released by the transport lock has at least one overhang embodied to prevent movement of the inner tube and the associated carrier containing the sensor element beyond the measuring position; and
the carrier is permanently connected, with a section of its outer shell facing away from the medium, to a medium-side section of the inner surface of the inner tube.

7. The electrochemical enzyme-based inline sensor according to claim 3, wherein the carrier permanently connected to the inner tube and containing the at least one sensor element is movable unidirectionally in the direction of the measuring medium in operation relative to a outer tube along a longitudinal axis of the sensor from an irradiation position to a hot steam sterilization position and from the hot steam sterilization position to a measuring position, and can be locked in the hot steam sterilization position and the measuring position.

8. The electrochemical enzyme-based inline sensor according to claim 1, wherein the enzyme is selected from:
an enzyme that catalyzes oxidation of lactate;
an enzyme that catalyzes oxidation of glutamate;
an enzyme that catalyzes oxidation of glutamine; and
an enzyme that oxidizes glucose.

9. The electrochemical enzyme-based inline sensor according to claim 1, wherein the inline sensor is designed as an amperometric inline sensor.

10. The electrochemical enzyme-based inline sensor according to claim 1, wherein the electrochemical inline sensor comprises at least two working electrodes, each comprising a sensor element, wherein the inline sensor is configured such that the at least two working electrodes can be introduced into the measuring medium simultaneously or successively.

11. The electrochemical enzyme-based inline sensor according to claim 1, further comprising a temperature sensor, wherein the temperature sensor is arranged within a sensor housing of the inline sensor, or is a separate temperature sensor electrically connected to the inline sensor.

12. An analytical system for process monitoring of a process or a bioprocess, comprising:
a reaction vessel including a cell culture contained in a suspension culture; and
at least one electrochemical enzyme-based inline sensor according to claim 1.

13. A method for continuously determining a total enzyme activity of an electrochemical enzyme-based inline sensor comprising:
providing a redox enzyme-based electrochemical inline sensor for process measurement technology or for bioprocess measurement technology to continuously monitor a state of an enzyme, comprising:
at least one sensor element comprising an amount of an immobilized redox enzyme, wherein the at least one sensor element is electrically coupled to a first electrode and/or is connected thereto, wherein the first electrode is configured as a working electrode, and
a counter electrode;
determining time-dependent charges received at a measuring region in a form of a current flow;
evaluating the current flow with an evaluation device to determine a concentration of an analyte in a liquid measuring medium; and
determining the total enzyme activity of the inline sensor via a measurand determined from the current flow, wherein the inline sensor is embodied to be installed in a process vessel and to be subsequently heat-sterilized.

14. The method according to claim 13, wherein the inline sensor further has a temperature sensor, wherein the temperature sensor is arranged within a sensor housing of the inline sensor or is a separate temperature sensor electrically connected to the inline sensor.

15. The method according to claim 13, wherein the measurand determined from the current flow is determined by:
converting the current flow at the working electrode into a sensor signal correlated with the amount of charge and compared with a reference signal that represents a maximum amount of charge of the inline sensor, or
comparing a measured current flow with a measured reference current.

16. The method according to claim 15, wherein a pre-alarm and/or an alarm is triggered at 90% or less than 90% of the reference signal, or at 90% or less than 90% of the maximum current flow, which indicates a need to replace the working electrode or the sensor element.

17. The method according to claim 13, wherein the redox enzyme-based inline sensor comprises:
at least one first electrode configured as a working electrode;
at least one second electrode configured as a counter electrode, wherein the at least one first electrode, at which an enzyme-based redox reaction takes place, is configured to detect and transmit generated electrical charges from starting operation of the inline sensor and to generate a charge analyte-dependent current flow resulting from the redox reaction; and
an evaluation device embodied to:
determine a concentration of one or more analytes from the generated charges; and
determine a measurand from the generated charges, wherein the measurand is used to determine an enzyme activity at the at least one first electrode, wherein the inline sensor is embodied to be installed in a process vessel and to be subsequently heat-sterilized.

18. The method according to claim 13, wherein a drop in current flow relative to a reference current or a saturation of a signal correlated with the amount of charge relative to a reference signal represents a decrease in the enzyme state of the inline sensor.

19. The method according to claim 18, wherein when the current flow drops relative to the reference current or when the signal correlated with the amount of charge becomes saturated relative to the reference signal, a further working electrode is introduced into the medium to be measured.

20. The electrochemical enzyme-based inline sensor according to claim 1, further comprising a third electrode configured as a reference electrode.
